(19) United States
(12) Patent Application Publication
(10) Pub. No.: US 2007/0093448 A1
(43) Pub. Date: Apr. 26, 2007

(54) Complexes that consist of vitamin D compounds or analogs thereof with a 5Z,7E,10(19)-triene system and methylated derivatives of beta-cyclodextrin

(76) Inventors: Juergen Westermann, Berlin (DE); Gabriele Winter, Schoenfliess (DE); Karsten Hoyer, Berlin (DE); Adrian Funke, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/398,577

(22) Filed: Apr. 6, 2006

Related U.S. Application Data
(60) Provisional application No. 60/673,360, filed on Apr. 21, 2005.

(30) Foreign Application Priority Data
Apr. 13, 2005 (DE) 102005017775.1

(51) Int. Cl. A61K 31/724; A61K 31/59; C08B 37/16

(52) U.S. Cl. 514/58; 514/167; 536/103

(57) ABSTRACT

The invention relates to complexes that consist of vitamin D compounds or analogs thereof with a 5Z,7E,10(19)-triene system and methylated derivatives of β-cyclodextrin, in particular a complex that consists of (thiazol-2-yl)-26,27-dinor-9,10-secocholesta-5,7,10(19)-triene-1,3,25-triol and heptakis-(2,6-di-O-methyl)-β-cyclodextrin (DIMEB).

This application claims the benefit of the filing date of U.S. Provisional Application Ser. No. 60/673,360 filed Apr. 21, 2006 which is incorporated by reference herein.

The invention relates to complexes that consist of vitamin D compounds or analogs thereof with a 5Z,7E,10(19)-triene system and methylated derivatives of the β-cyclodextrin, in particular a complex that consists of (thiazol-2-yl)-26,27-dinor-9,10-secocholesta-5,7,10(19)-triene-1,3,25-triol and heptakis-(2,6-di-O-methyl)-β-cyclodextrin (DIMEB).

The application is based on the following definitions of terms:

Analogs: Analogs are compounds of a natural substance that are structurally related to a guide structure and that have, at least qualitatively, the same physiological action as the guide structure.

Natural Substances: Natural substances are compounds that occur naturally in plants, animals and/or humans and are isolated therefrom.

Vitamin D Compounds: Vitamin D compounds are a fat-soluble group of natural substances, derived from 7,8-didehydrosterols by photochemical ring opening and isomerization, with more or less pronounced anti-rachitic action. The vitamin D compounds include, for example,
calciferols such as cholecalciferol (vitamin D₃, calciol), ergocalciferol, (vitamin D₂, ercalciol) and
Vitamin D precursors such as 7-dehydrocholesterol (7,8-didehydrocholesterol, tacalciol, provitamin D₃, procalciol, procholecalciferol) and ergosterol (ertacalciol, provitamin D₂) and
Vitamin D metabolites such as calcifediol (25-hydroxycholecalciferol, 25-hydroxy vitamin D₃, calcidiol, pro-hormone), calcitriol (1α,25-dihydroxycholecalciferol, soltriol, 1α,25-dihydroxy-vitamin D₃) and 24R,25-dihydroxycholecalciferol (hydroxycalcidiol), ercalcidiol (25-hydroxyergocalciferol), ercalcitriol (1α,25-dihydroxyergocalciferol, 1α,25-dihydroxy-vitamin D₂), 1α-hydroxycholecalciferol
   Vitamin D Analogs: The vitamin D analogs are molecules with a 5Z,7E,10(19)-triene system.
   Ratio of Use: Ratio of use is the molar ratio of educts (vitamin D compound or analogs thereof to cyclodextrin) in the complex production.
   Mean Molar Ratio: Refers to the number of moles of vitamin D or vitamin D derivative to the number of moles of cyclodextrin derivative in a sample of the complex with a purity of the complex of at least 98% (relative to the weights).
   Cyclodextrin: Cyclodextrin is used as a generic term for all native and modified cyclodextrins.
   Cyclodextrin Derivatives: Cyclodextrin derivatives are compounds in which the glucopyranose units in each case can be substituted in one or more of the positions 2-O, 3-O and/or 6-O. The substitution can be partial or complete, symmetrical or unsymmetrical, uniform at each glucopyranose unit or different. Cyclodextrin derivatives are also named modified cyclodextrins below. Cyclodextrin derivatives can have 6, 7 or 8 glucopyranose units.
   DIMEB: Heptakis-(2,6-di-O-methyl)-β-cyclodextrin.
   TRIMEB: Permethylated β-cyclodextrin with 21 methyl groups as substituents.
   RAMEB: Statistically methylated β-cyclodextrin (randomly methylated β-cyclodextrin, MW 1303).

DIMEB, TRIMEB and RAMEB are commercially available with degrees of purity of up to 98% and more.

Vitamin D compounds and analogs thereof are in some cases highly effective substances that are used in a low therapeutic dose. The vitamin D compounds include calcitriols. As pure substances and in formulations, these substances are sensitive to atmospheric oxygen, temperature and light.

Cyclodextrins consist of 6-, 7- or 8-membered cyclically-arranged glucopyranose units, optionally derivatized, which are glycosidically bound via the 1-O or 4-O position. These cyclic glucopyranose oligosaccharides are referred to as α, β or γ-cyclodextrins (so-called Schardinger cyclodextrins). Cyclodextrins are produced from starch by, e.g., enzymatic means (Szejtly, J., Cyclodextrin Technology, Davies, J. E., Ed; Kluwer Academic Press, Dordrecht, The Netherlands 1988).

It is known to use cyclodextrins in pharmaceutical compositions. Because of their circular structure, cyclodextrins have a hydrophilic exterior and a hydrophobic inside pocket. By encasing in particular hydrophobic areas of the molecule, cyclodextrins can achieve a “molecular encapsulation” or “masking” of active ingredients that is used as, for example, protective encasing of sensitive molecules in cosmetic and pharmaceutical formulations. As a result, improved solubilities of substances and even reduced toxicities can be achieved.

Native and modified cyclodextrins are distinguished. Native cyclodextrins are the α-, β- and γ-cyclodextrins that are accessible to the enzyme glucotransferase from starch. The high and low-membered rings do not play any significant role in practice.

Depending on substitution and degree of substitution, the modified cyclodextrins have varying properties. By introducing polar groups, their water solubility is increased; the complexes are then also often better water-soluble. The bioavailability of less water-soluble active substances can thus be improved. By way of example, hydroxyalkyl derivatives, such as, e.g., hydroxypropyl-β-cyclodextrin (HP-β-CD), can be mentioned here.

Methylated cyclodextrins with a degree of methylation <2, which are used for the production of complexes, are described in U.S. Pat. No. 6,602,860. In U.S. Pat. No. 4,727,064, O-alkyl-substituted cyclodextrins are disclosed for complexing active ingredients.

The amorphous methyl cyclodextrin is commercially available under the cyclodextrins that are methyl-substituted in one place. The latter tends only slightly toward forming crystalline complexes. For the most part, amorphous solid complexes or soluble complexes that stabilize the active ingredient are formed with amorphous methyl-cyclodextrin.

DIMEB has other properties. Here, this is a crystalline product. The tendency toward formation of inclusion complexes increases with increasing lipophilia (Rekharsky, M.; Inoue, Y.; Chem. Rev. 1998, 98, 1875-1918).

For example, the bibliographic reference EP121777 discloses the production of DIMEB. Starting from β-cyclodextrin, the hydroxy groups in 2- and 6-positions are selectively methylated. The product is purified by crystallization. In the literature, DIMEB had been described as early as 1968 (Tetrahedron 1968, 24, 803-806). DIMEB has a high affinity for lipophilic substances. DIMEB has, i.a., a high affinity for cholesterol, so that cholesterol is extracted from the blood cell membranes in the organism. In intravenous administration, DIMEB shows hemolytic effects and forms precipitates with cholesterol. DIMEB is very readily soluble in water. In addition, DIMEB has a negative temperature effect; it is very readily soluble at 20° C. (>50 g/100 ml of water) and not very soluble while being heated (<1 g/100 ml). At 20° C., DIMEB is present as a hydrated, readily soluble form; at higher temperatures, a less readily soluble anhydrate is formed (Saenger et al., Langmuir 2002, 18, 5974-5976).

In free form as an excipient, DIMEB can crystallize out because of the lower solubility while being heated during sterilization. The uniform substitution with DIMEB has the effect that it itself and also as a complex can be readily crystallized, and the complexes generally are less readily soluble than DIMEB itself.

In the group of cyclodextrins and cyclodextrin derivatives, there are a number of compounds with properties for dissolving that are comparable to DIMEB, some with partially better properties for dissolving than DIMEB.

Modified cyclodextrins with an unsymmetrical substitution and/or a non-uniform substitution and also the complexes generally show a lower crystallinity than modified cyclodextrins with symmetrical and/or uniform substitution. Thus, for example, the statistically methylated β-cyclodextrin (RAMEB) is readily water-soluble, and also the complexes of the RAMEBs tend only slightly toward crystallization.

Cyclodextrin complexes of vitamin D compounds or analogs thereof are partially known. An attempt was made to improve the stability and the solubility by complexing (Pharmazie [Pharmaceutics] 1993, 35, 779-787, GB-A 2,037,773).

Vitamin D compounds and analogs thereof are, for example, the natural calcitriol (1α-25-dihydroxyvitamin D₃), the calcifediol (25-hydroxyvitamin D₃), the cholecalciferol (vitamin D₃), the calcipotriol (CAS 112965-21-6) and the tacalcitol (CAS-57333-96-7).

Additional analogs of the vitamin D series are disclosed in WO 94/07853, WO 01/07405 and WO 97/41096.

A compound according to WO 01/07405 is (thiazol-2-yl)-26,27-dinor-9,10-secocholesta-5,7,10(19)-triene-1,3,25-triol (compound 1).

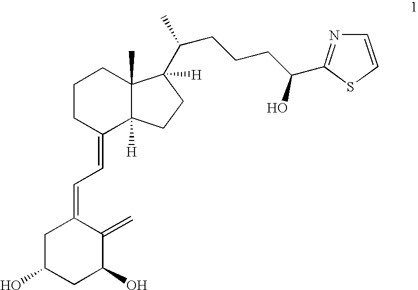

This calcitriol derivative is developed for treatment of psoriasis with topical and oral administration.

Vitamin D compounds and most analogs thereof have instability toward air, temperature and exposure to light, as do many compounds of WO 01/07405 and WO 97/41096.

After four months, a sample of compound 1, stored at room temperature, shows, for example, only a content of 60% at a previtamin content of about 2% in comparison to samples that are freshly produced or samples that are stored under inert conditions.

Vitamin D compounds or analogs thereof often have a 5Z,7E,10(19)-triene system, which, moreover, tends to isomerize to the previtamin. In solution, these substances are subject to a natural isomerization to the previtamin because of a sigmatropic 1,7-H shift (Curtin, M. L.; Okamura, W. H.: J. Am. Chem. Soc. 1991, 113, 6958-6966). Affected by this are vitamin D compounds and analogs thereof with a 5Z,7E,10(19)-triene system. The natural equilibrium is at a ratio of about 93:7 depending on solvent, temperature, concentration and service life in solution. The cis form of the vitamin form is thermodynamically favorable (Havinga, E. Experentia, 1973, 29, 1181). In contrast to calcitriols that are derived from steroids, 19-nor-calcitriols do not show this isomerization (De Clerk, P. J. et al., J. Med. Chem. 1999, 42, 3539-3556).

For example, the compound 1 isomerizes to the compound 2.

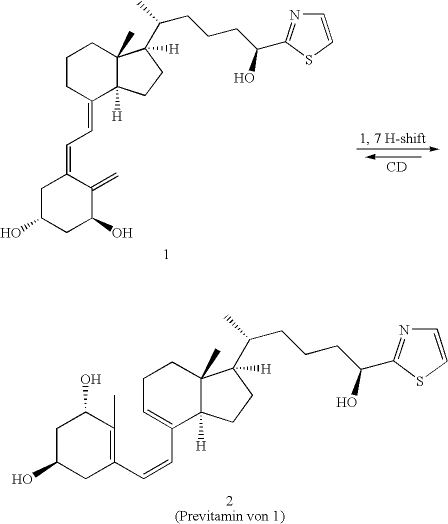

In Patent Application WO 97/23242, complexes of isopropyl (5Z,7E,22E)-(1S,3R,24R)-1,3,24-trihydroxy-9,10-secocholesta-5,7,10(19),22-tetraene-25-carboxylate are disclosed. In this application, the isomerization to the previtamin in particular is not discussed in more detail.

In 1980, Szejtli et al. described the vitamin D₃-β-cyclodextrin complex (Szejtly et al., Pharmazie 1980, 35, 779). The production is carried out in ethanolic solution (GB 2037773). An influence of the vitamin-previtamin equilibrium in connection with the complexing is not mentioned.

In the literature, references to the isomerization of vitamin D to previtamin D under the influence of β-cyclodextrin can be found (Tian, X. Q.; Hollick, M. F.; J. Biolog. Chem., 1995, 270, 8706-8711). This publication is based on the in vivo isomerization of the previtamin to the vitamin, which was tested as a model for the biomimetic isomerization. The previtamin-vitamin isomerization occurs in the photolytic biogenesis of vitamin D₃ after a pericyclic ring opening of the 7-dehydrocholesterol in the triene system. In the photolytic opening of the steroid (provitamin), a secosteroid is produced that first is present as a previtamin after the cleavage of the bond at C9 and C10 and then isomerizes to the vitamin. Because of the reversibility of this isomerization, both isomers are present together in solution.

Studies on isomerization are described in the literature (Curtin, M. L., Okamura, W. H., J. Am. Chem. Soc. 1991, 113, 6958-6966). Supplementary literature for previtamin formation of vitamin D compounds is found at the following sites: J. Am. Chem. Soc. 113, 6958ff (1991); J. Biol. Chem. 270, 8706ff (1995); J. Am. Chem. Soc. 121, 4933 (1999); Tetrahedron Lett. 33, 5445ff (1992); J. Bone Miner. Res. 8, 1009 ff (1993); J. Med. Chem. 37, 2387ff (1994); BBRC 189, 1450ff (1992); J. Biol. Chem. 268, 13811 (1993); J. Biol. Chem. 268, 2022 (1993).

Example 1 of this application confirms that vitamin D₂ and calcitriol are subject to an isomerization to the previtamin in the complexing with native cyclodextrins.

The presence of the active ingredient in isomer forms is not desirable. In the case of low-dose active ingredients for oral or systemic administration, fluctuations unavoidably occur in the active ingredient concentration (insufficient content uniformity), which have a greater impact, the lower the dose of the active ingredient that is present.

The object of this invention is to stabilize vitamin D compounds or analogs thereof with a 5Z,7E,10(19)-triene system relative to atmospheric oxygen, temperature and light without an isomerization to the previtamin taking place.

To solve this technical problem, the invention teaches a complex that consists of a vitamin D compound or a vitamin D analog with a cyclodextrin derivative, whereby optionally the mean molar ratio of vitamin D to the cyclodextrin derivative is in the range of 1:5 to 5:1, in particular 1:2 to 2:1, preferably 1:1.2 to 1.2:1.

The cyclodextrin derivative preferably contains n=6 or 7 glucopyranose units. In addition, a cyclodextrin derivative is preferred, whereby R1 is a 6-O substituent, R2 is a 4-O substituent, and R3 is a 2-O substituent of the glucopyranose units (whereby the term m-O substituent means that the hydrogen on the oxygen atom, which is bonded to the carbon atom with the numbering scheme m of a glucopyranose unit, is replaced by the relevant substituents), whereby R1, R2 and R3 can be the same or different and are any physiologically compatible radical, but not —H at the same time, and are preferably —H, C1-C8-alkyl, linear or branched, saturated or unsaturated, —SO₂OH, —PO(OH)₂, or —CO—R4 with R4=C1-C8-alkyl, whereby the C1-C8-alkyl can be substituted in one or more places, on the same or on different C atoms with —OH, —COOH, —CONHR5, —NHCOR6, —SO₂OH, —PO(OH)₂ or tetrazol-5-yl, with R5=—H or C1-C4-alkyl and R6=carboxylphenyl, whereby R1, R2, and R3 can be randomized in various glucopyranose units, whereby an oxygen atom or several oxygen atoms of the glucopyranose units, in particular the oxygen atom 6-O, can be replaced by sulfur atoms, or with a physiologically compatible salt of such a cyclodextrin derivative. Especially preferred is a cyclodextrin derivative, whereby n=7 and R1, R2, and R3 are the same or different but are not —H at the same time, and are —H or C1-C8-alkyl or C1-C8-hydroxyalkyl.

In particular, methyl, ethyl, propyl, isopropyl, butyl, isobutyl, tert-butyl, pentyl, etc., fall under C1-C8 alkyl. C1-C8 Hydroxyalkyl is based on C1-C8 alkyl, whereby one or more, preferably one, of the C atoms carries a hydroxyl group or several hydroxyl groups.

The object of this invention is further achieved by complexing of the vitamin D compounds and analogs thereof with methylated derivatives of the β-cyclodextrin.

Suitable methylated derivatives of the β-cyclodextrin are in particular heptakis-(2,6-di-O-methyl)-β-cyclodextrin (DIMEB), statistically methylated β-cyclodextrin (RAMEB) and permethylated β-cyclodextrin with 21 methyl groups (TRIMEB).

The induction of an isomerization to the previtamin as in the case of native cyclodextrins virtually does not occur with these methylated β-cyclodextrin derivatives.

The isomerization of vitamin D compounds and analogs thereof to the previtamin that is induced by native cyclodextrins thus can be avoided for all vitamin D compounds and analogs thereof with a 5Z,7E,10(19)-triene system by means of methylated β-cyclodextrin derivatives. This means that with methylated β-cyclodextrin derivatives, these compounds can be stabilized relative to atmospheric oxygen, temperature and light without the drawbacks of an isomerization to the previtamin.

Vitamin D analogs of general formula I, 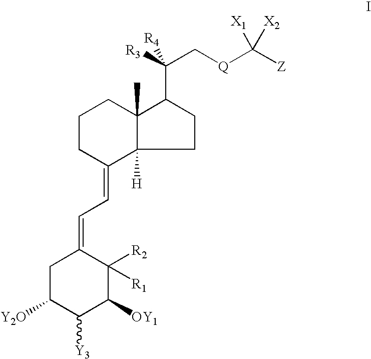
in which
Y₁ and Y₂, independently of one another, each mean a hydrogen atom or a group —C(O)R₅,
and Y₃ means a hydrogen atom or a hydroxy group, a halogen atom, a group —OC(O)R₅ or an OR₅ group,
   whereby
   R₅ stands for an aromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms, and/or 1-2 NH groups and/or optionally is substituted by 1-2 hydroxy groups, 1-2 amino groups, 1-2 SH groups, 1-2 COOH groups and/or 1-2 phenyl groups,
   and the group Y₃ can be present both in the 2α-situation and in the epimeric 2β-situation,
R₁ and R₂ together mean an exocyclic methylene group,
R₃ and R₄, independently of one another, each mean a hydrogen atom, a fluorine, chlorine or bromine atom, an alkyl group with 1 to 4 carbon atoms, together a methylene group, or, together with the quaternary carbon atom 20, a 3- to 7-membered, saturated or unsaturated carbocyclic ring,
Q means a straight-chain alkylene group with 1 to 5 carbon atoms,
X₁ and X₂ together mean a double-bonded keto-oxygen atom or, independently of one another, a hydrogen atom, a hydroxy group, an —O(CO)R₅ group, a fluorine, chlorine or bromine atom,
   whereby X₁ and X₂ should each be, not simultaneously, a hydroxy group or an —O(CO)R₅ group,
Z means a carbocyclic or heterocyclic, optionally aromatic or heteroaromatic ring with 5 or 6 ring members or a condensed ring system that consists of a 5- and a 6-membered ring or two 6-membered rings, which can be substituted by one or more fluorine, chlorine, bromine or iodine atoms, one or more hydroxy groups, one or more COOR₆ groups, one or more C₁-C₅-alkyl groups, which in turn can be substituted by one or more fluorine, chlorine, bromine, or iodine atoms, C₁-C₆-alkoxy groups and/or COOR₆ groups,
   whereby
R₆ stands for a C₁-C₆-alkyl group, a benzyl group or a phenyl group,
   as well as all possible epimers or diastereomers and mixtures thereof,
   can [be] especially advantageous.

These vitamin D analogs are disclosed in WO 01/07405. Also, however, vitamin D analogs according to the bibliographic references WO 97/00242, WO 97/41096, and WO 99/16745 can be used. All of these bibliographic references are herewith “incorporated by reference.”

The following vitamin D compounds and analogs thereof can also be stabilized as cyclodextrin derivative complexes, in particular as DIMEB, RAMEB or TRIMEB complexes:

Calcitriol [(5Z,7E)-(1S,3R)-9,10-secocholesta-5,7,10(19)-triene-1,3,25-triol, CAS No. 32222-66-3)], calcifediol [25-hydroxy-vitamin D₃ (CAS No. 19356-17-3)], cholecalciferol [vitamin D₃, (CAS-No. 67-97-0)]; ergocalciferol [vitamin D₂, (5Z,7E,22E)-(3S)-9,10-secoergosta-5,7,10(19),22-tetraen-3-ol, (CAS No. 50-14-6)]; 1α-hydroxycholecalciferol [(1alpha,3beta,5Z,7E)-9,10-secocholesta-5,7,10(19)-triene-1,3-diol, (CAS No. 41294-56-8)], calcipotriol [(1S,3R,5Z,7E,22E,24S)-24-cyclopropyl-9,10-secochola-5,7,10(19), 22-tetraene-1,3,24-triol, (CAS 112965-21-6)], tacalcitol [(+)-(5Z,7E,24R)-9,10-secocholesta-5,7,10(19)-triene-1alpha,3beta-24-triol monohydrate, (CAS No. 57333-96-7)].

Also, the following compounds are suitable:
(7E)-(1R,3R,24aR)-24a-(Oxazol-4-yl)-24a-homo-19-nor-9,10-secochola-5,7-diene-1,3,24a-triol
(7E)-(1R,3R,24aS)-24a-(Oxazol-4-yl)-24a-homo-19-nor-9,10-secochola-5,7-diene-1,3,24a-triol
(5Z,7E)-(1S,3R,24aR)-24a-(Oxazol-4-yl)-24a-homo-9,10-secochola-5,7,10(19)-triene-1,3,24a-triol
(5Z,7E)-(1S,3R,24aS)-24a-(Oxazol-4-yl)-24a-homo-9,10-secochola-5,7,10(19)-triene-1,3,24a-triol
(7E)-(1R,3R,24aR)-24a-(Thiazol-2-yl)-24a-homo-19-nor-9,10-secochola-5,7-diene-1,3,24a-triol
(7E)-(1R,3R,24aS)-24a-(Thiazol-2-yl)-24a-homo-19-nor-9,10-secochola-5,7-diene-1,3,24a-triol
(7E)-(1R,3R)-1,3-Dihydroxy-24a-(thiazol-2-yl-)-24-a-homo-19-nor-9,10-secochola-5,7-dien-24a-one
(5Z 7E)-(1S,3R,24aR)-24a-(Thiazol-2-yl)-24a-homo-9,10-secochola-5,7,10(19)-triene-1,3,24a-triol
(5Z,7E)-(1S,3R,24aS)-24a-(Thiazol-2-yl)-24a-homo-9,10-secochola-5,7,10(19)-triene-1,3,24a-triol
(5Z 7E)-(1S,3R)-1,3-Dihydroxy-24a-(thiazol-2-yl)-24a-homo-9,10-secochola-5,7,10(19)-trien-24a-one
(7E)-(1R,3R,24aR)-24a-(4-Methylthiazol-2-yl)-24a-homo-19-nor-9,10-secochola-5,7-diene-1,3,24a-triol
(7E)-(1R,3R,24aS)-24a-(4-Methylthiazol-2-yl)-24a-homo-19-nor-9,10-secochola-5,7-diene-1,3,24a-triol
(7E)-(1R,3R)-1,3-Dihydroxy-24a-(4-methylthiazol-2-yl)-24a-homo-19-nor-9,10-secochola-5,7-dien-24a-one
(5Z,7E)-(1S,3R,24aR)-24a-(4-Methylthiazol-2-yl)-24a-homo-9,10-secochola-5,7,10(19)-triene-1,3,24a-triol
(5Z,7E)-(1S,3R,24aS)-24a-(4-Methylthiazol-2-yl)-24a-homo-9,10-secochola-5,7,10(19)-triene-1,3,24a-triol
(5Z,7E)-(1S,3R)-1,3-Dihydroxy-24a-(4-methylthiazol-2-yl)-24a-homo-9,10-secochola-5,7,10(19)-trien-24a-one
(7E)-(1R,3R,24aR)-24a-(Thien-2-yl)-24a-homo-19-nor-9,10-secochola-5,7-diene-1,3,24a-triol
(7E)-(1R,3R,2aS)-24a-(Thien-2-yl)-24a-homo-19-nor-9,10-secochola-5,7-diene-1,3,24a-triol
(7E)-(1R,3R)-1,3-Dihydroxy-24a-(thien-2-yl)-24a-homo-19-nor-9,10-secochola-5,7-dien-24a-one
(5Z,7E)-(1S,3R,24aR)-24a-(Thien-2-yl)-24a-homo-9,10-secochola-5,7,10(19)-triene-1,3,24a-triol
(5Z,7E)-(1S,3R,24aS)-24a-(Thien-2-yl)-24a-homo-9,10-secochola-5,7,10(19)-triene-1,3,24a-triol
(7E)-(1R,2S,3R,24aR)-24a-Thien-2-yl-24a-homo-19-nor-9,10-secochola-5,7-diene-1,2,3,24-tetrol
(7E)-(1R,2S,3R,24aS)-24a-Thien-2-yl-24a-homo-19-nor-9,10-secochola-5,7-diene-1,2,3,24-tetrol
(7E)-(1R,2S,3R)-24a-Thien-2-yl-1,2,3-trihydroxy-24a-homo-9,10-secochola-5,7-dien-24a-one
(7E)-(1R,3R,24aR)-24a-(4-Methylthien-2-yl)-24a-homo-19-nor-9,10-secochola-5,7-diene-1,3,24a-triol
(7E)-(1R,3R,24aS)-24a-(4-Methylthien-2-yl)-24a-homo-19-nor-9,10-secochola-5,7-diene-1,3,24a-triol
(7E)-(1R,3R)-1,3-Dihydroxy-24a-(4-methylthien-2-yl)-24a-homo-19-nor-9,10-secochola-5,7-dien-24a-one
(5Z,7E)-(1S,3R,24aR)-24a-(4-Methylthien-2-yl)-24a-homo-9,10-secochola-5,7,10(19)-triene-1,3,24a-triol
(5Z 7E)-(1S,3R,24aS)-24a-(4-Methylthien-2-yl)-24a-homo-9,10-secochola-5,7,10(19)-triene-1,3,24a-triol
(5Z,7E)-(1S,3R)-1,3-Dihydroxy-24a-(4-methylthien-2-yl)-24a-homo-9,10-secochola-5,7,10(19)-trien-24a-one
(7E)-(1R,2S,3R,24aR)-24a-(4-Methylthien-2-yl)-24a-homo-19-nor-9,10-secocola-5,7-diene-1,2,3,24-tetrol
(7E)-(1R,2S,3R,24aS)-24a-(4-Methylthien-2-yl)-24a-homo-19-nor-9,10-secochola-5,7-diene-1,2,3,24-tetrol
(7E)-(1R,2S,3R)-24a-(4-Methylthien-2-yl)-1,2,3-trihydroxy-24a-homo-19-nor-9,10-secochola-5,7-dien-24a-one
(7E)-(1R,3R,24aR)-24a-(5-Ethylthien-2-yl)-24a-homo-19-nor-9,10-secochola-5,7-diene-1,3,24a-triol
(7E)-(1R,3R,24aS)-24a-(5-Ethylthien-2-yl)-24a-homo-19-nor-9,10-secochola-5,7-diene-1,3,24a-triol
(7E)-(1R,3R,24aR)-24a-[5-(2-Hydroxyethyl)-4-methylthiazol-2-yl]-24a-homo-19-nor-9,10-secochola-5,7-diene-1,3,24a-triol
(7E)-(1R,3R,24aS)-24a-[5-(2-Hydroxyethyl)-4-methylthiazol-2-yl]-24a-homo-19-nor-9,10-secochola-5,7-diene-1,3,24a-triol
(7E)-(1R,3R)-1,3-Dihydroxy-24a-[5-(2-hydroxyethyl)-4-methylthiazol-2-yl]-24a-homo-19-nor-9,10-secochola-5,7-dien-24a-one
(7E)-(1R,3R,24aR)-24a-(Benzothiazol-2-yl)-24a-homo-19-nor-9,10-secochola-5,7-diene-1,3,24a-triol
(7E)-(1R,3R,24aS)-24a-(Benzothiazol-2-yl)-24a-homo-19-nor-9,10-secochola-5,7-diene-1,3,24a-triol
(7E)-(1R,3R)-24a-(Benzothiazol-2-yl)-1,3-dihydroxy-24a-homo-19-nor-9,10-secochola-5,7-dien-24a-one
(7E)-(1R,3R,24aR)-24a-(Benzofuran-2-yl)-24a-homo-19-nor-9,10-secochola-5,7-diene-1,3,24a-triol
(7E)-(1R,3R,24aS)-24a-(Benzofuran-2-yl)-24a-homo-19-nor-9,10-secochola-5,7-diene-1,3,24a-triol
(7E)-(1R,3R)-24a-(Benzofuran-2-yl)-1,3-dihydroxy-24a-homo-19-nor-9,10-secochola-5,7-dien-24a-one
(7E)-(1R,3R,24aR)-24a-(Benzothiophen-2-yl)-24a-homo-19-nor-9,10-secochola-5,7-diene-1,3,24a-triol
(7E)-(1R,3R,24aS)-24a-(Benzothiophen-2-yl)-24a-homo-19-nor-9,19-secochola-5,7-diene-1,3,24a-triol
(7E)-(1R,3R)-24a-(Benzothiophen-2-yl)-1,3-dihydroxy-24a-homo-19-nor-9,10-secochola-5,7-dien-24a-one
(7E)-(1R,3R,24aR)-24a-(1-Methylbenzimidazol-2-yl)-19-nor-9,10-secochola-5,7-diene-1,3,24a-triol
(7E)-(1R,3R,24aS)-24a-(1-Methylbenzimidazol-2-yl)-19-nor-9,10-secochola-5,7-diene-1,3,24a-triol
(7E)-(1R,3R)-1,3-Dihydroxy-24a-(1-methylbenzimidazol-2-yl)-24a-homo-19-nor-9,10-secochola-5,7-dien-24a-one
(7E)-(1R,3R)-1-(1,3-Dihydroxy-24a-homo-19-nor-9,10-secochola-5,7-dien-24a-yl)-3-[(4-methoxyphenyl)methoxy]-1H-pyrazole-4-carboxylic acid ethyl ester
(7E)-(1R,3R)-1-(1,3-Dihydroxy-24a-homo-19-nor-9,10-secochola-5,7-dien-24a-yl)-3-hydroxy-1H-pyrazole-4-carboxylic acid ethyl ester
(7E)-(1R,3R,24aR)-24a-(4-Methylphenyl)-24a-homo-19-nor-9,10-secochola-5,7-diene-1,3,24a-triol
(7E)-(1R,3R,24aS)-24a-(4-Methylphenyl)-24a-homo-19-nor-9,10-secochola-5,7-diene-1,3,24a-triol
(7E)-(1R,3R)-1,3-Dihydroxy-24a-(4-methylphenyl)-24a-homo-19-nor-9,10-secochola-5,7-dien-24a-one
(7E)-(1R,2R,3R,24aR)-24a-(4-Methylphenyl)-24a-homo-19-nor-9,10-secochola-5,7-diene-1,2,3,24a-tetrol
(7E)-(1R,RS,3R,24aS)-24a-(4-Methylphenyl)-24a-homo-19-nor-9,10-secochola-5,7-diene-1,2,3,24a-tetrol
(7E)-(1R,3R,24aR)-24a-(4-Trifluoromethylphenyl)-19-nor-9,10-secochola-5,7-diene-1,3,24a-triol
(7E)-(1R,3R,24aS)-24a-(4-Trifluoromethylphenyl)-19-nor-9,10-secochola-5,7-diene-1,3,24a-triol
(7E)-(1R,3R)-1,3-Dihydroxy-24a-(4-trifluoromethylphenyl)-24a-homo-19-nor-9,10-secochola-5,7-dien-24a-one
(7E)-(1R,3R,24aR)-24a-(4-Methoxyphenyl)-24a-homo-19-nor-9,10-secochola-5,7-diene-1,3,24a-triol
(7E)-(1R,3R,24aS)-24a-(4-Methoxyphenyl)-24a-homo-19-nor-9,10-secochola-5,7-diene-1,3,24a-triol
(7E)-(1R,3R)-1,3-Dihydroxy-24a-(4-methoxyphenyl)-24a-homo-19-nor-9,10-secochola-5,7-dien-24a-one
(7E)-(1R,3R,20S,24aR)-24a-(Thiazol-2-yl)-24a-homo-19-nor-9,10-secochola-5,7-diene-1,3,24a-triol
(7E)-(1R,3R,20S,24aS)-24a-(Thiazol-2-yl)-24-a-homo-19-nor-9,10-secochola-5,7-diene-1,3,24a-triol
(7E)-(1R,3R,20S)-1,3-Dihydroxy-24a-(thiazol-2-yl)-24-a-homo-19-nor-9,10-secochola-5,7-dien-24a-one
(5Z,7E)-(1S,3R,20S,24aR)-24a-(Thiazol-2-yl)-24a-homo-9,10-secochola-5,7,10(19)-triene-1,3,24a-triol
(5Z,7E)-(1S,3R,20S,24aS)-24a-(Thiazol-2-yl)-24a-homo-9,10-secochola-5,7,10(19)-triene-1,3,24a-triol
(5Z,7E)-(1S,3R,20S)-1,3-Dihydroxy-24a-(thiazol-2-yl)-24a-homo-9,10-secochola-5,7,10(19)-trien-24a-one
(5Z,7E)-(1S,3R,24S)-24-(Thiazol-2-yl)-9,10-secochola-5,7,10(19)-triene-1,3,24-triol
(5Z,7E)-(1S,3R,24R)-24-(Thiazol-2-yl)-9,10-secochola-5,7,10(19)-triene-1,3,24-triol
(5Z 7E)-(1S,3R)-1,3-Dihydroxy-24-(thiazol-2-yl)-9,10-secochola-5,7,10(19)-trien-24-one
(7E)-(1R,3R,20S,24aR)-24a-(Oxazol-4-yl)-24a-homo-19-nor-9,10-secochola-5,7-diene-1,3,24a-triol
(7E)-(1R,3R,20S,24aS)-24a-(Oxazol-4-yl)-24-a-homo-19-nor-9,10-secochola-5,7-diene-1,3,24a-triol
(7E)-(1R,3R,20S)-1,3-Dihydroxy-24a-(oxazol-4-yl)-24-a-homo-19-nor-9,10-secochola-5,7-dien-24a-one
(5Z,7E)-(1S,3R,20S,24aR)-24a-(Oxazol-4-yl)-24a-homo-9,10-secochola-5,7,10(19)-triene-1,3,24a-triol
(5Z 7E)-(1S,3R,20S,24aS)-24a-(Oxazol-4-yl)-24a-homo-9,10-secochola-5,7,10(19)-triene-1,3,24a-triol
(5Z,7E)-(1S,3R,20S)-1,3-Dihydroxy-24a-(oxazol-4-yl)-24a-homo-9,10-secochola-5,7,10(19)-trien-24a-one
(5Z,7E)-(1S,3R,24R)-24-(Oxazol-4-yl)-9,10-secochola-5,7,10(19)-triene-1,3,24a-triol
(5Z,7E)-(1S,3R,24S)-24-(Oxazol-4-yl)-9,10-secochola-5,7,10(19)-triene-1,3,24a-triol
(5Z 7E)-(1S,3R)-1,3-Dihydroxy-24-(oxazol-4-yl)-9,10-secochola-5,7,10(19)-trien-24-one
(7E)-(1R,3R,20S,24aR)-24a-(4-Methylthiazol-2-yl)-24a-homo-19-nor-9,10-secochola-5,7-diene-1,3,24a-triol
(7E)-(1R,3R,20S,24aS)-24a-(4-Methylthiazol-2-yl)-24a-homo-19-nor-9,10-secochola-5,7-diene-1,3,24a-triol
(7E)-(1R,3R,20S)-1,3-Dihydroxy-24a-(4-methylthiazol-2-yl)-24-a-homo-19-nor-9,10-secochola-5,7-dien-24a-one
(5Z,7E)-(1S,3R,20S,24aR)-(4-Methylthiazol-2-yl)-24a-homo-9,10-secochola-5,7,10(19)-triene-1,3,24a-triol
(5Z,7E)-(1S,3R,20S,24aS)-24a-(4-Methylthiazol-2-yl)-24a-homo-9,10-secochola-5,7,10(19)-triene-1,3,24a-triol
(5Z,7E)-(1S,3R,20)-1,3-Dihydroxy-24a-(4-methylthiazol-2-yl)-24a-homo-9,10-secochola-5,7,10(19)-trien-24a-one
(5Z,7E)-(1S,3R,24R)-24-(4-Methylthiazol-2-yl)-9,10-secochola-5,7,10(19)-triene-1,3,24a-triol
(5Z,7E)-(1S,3R,24S)-24-(4-Methylthiazol-2-yl)-9,10-secochola-5,7,10(19)-triene-1,3,24a-triol
(5Z,7E)-(1S,3R)-1,3-Dihydroxy-24-(4-methylthiazol-2-yl)-9,10-secochola-5,7,10(19)-trien-24-one
(7E)-(1R,3R,20S,24aR)-24a-(4-Methylthien-2-yl)-24a-homo-19-nor-9,10-secochola-5,7-diene-1,3,24a-triol
(7E)-(1R,3R,20S,24aS)-24a-(4-Methylthien-2-yl)-24-a-homo-19-nor-9,10-secochola-5,7-diene-1,3,24a-triol
(7E)-(1R,3R,20S)-1,3-Dihydroxy-24a-(4-methylthien-2-yl)-24-a-homo-19-nor-9,10-secochola-5,7-dien-24a-one
(5Z,7E)-(1S,3R,20S,24aR)-24a-(4-Methylthien-2-yl)-24a-homo-9,10-secochola-5,7,10(19)-triene-1,3,24a-triol
(5Z,7E)-(1S,3R,20S,24aS)-24a-(4-Methylthien-2-yl)-24a-homo-9,10-secochola-5,7,10(19)-triene-1,3,24a-triol
(5Z,7E)-(1S,3R,20S)-1,3-Dihydroxy-24a-(4-methylthien-2-yl)-24a-homo-9,10-secochola-5,7,10(19)-trien-24a-one
(5Z,7E)-(1S,3R,24R)-24-(4-Methylthien-2-yl)-9,10-secochola-5,7,10(19)-triene-1,3,24a-triol
(5Z,7E)-(1S,3R,24S)-24-(4-Methylthien-2-yl)-9,10-secochola-5,7,10(19)-triene-1,3,24a-triol
(5Z,7E)-(1S,3R)-1,3-Dihydroxy-24-(4-methylthien-2-yl)-9,10-secochola-5,7,10(19)-trien-24-one
(7E)-(1R,3R,20S,24aR)-24a-(Thien-2-yl)-24a-homo-19-nor-9,10-secochola-5,7-diene-1,3,24a-triol
(7E)-(1R,3R,20S,24aS)-24a-(Thien-2-yl)-24-a-homo-19-nor-9,10-secochola-5,7-diene-1,3,24a-triol
(7E)-(1R,3R,20S)-1,3-Dihydroxy-24a-(thien-2-yl)-24-a-homo-19-nor-9,10-secochola-5,7-dien-24a-one
(5Z,7E)-(1S,3R,20S,24aR)-24a-(Thien-2-yl)-24a-homo-9,10-secochola-5,7,10(19)-triene-1,3,24a-triol
(5Z,7E)-(1S,3R,20S,24aS)-24a-(Thien-2-yl)-24a-homo-9,10-secochola-5,7,10(19)-triene-1,3,24a-triol
(5Z,7E)-(1S,3R,20S)-1,3-Dihydroxy-24a-(thien-2-yl)-24a-homo-9,10-secochola-5,7,10(19)-trien-24a-one
(5Z,7E)-(1S,3R,24R)-24-(Thien-2-yl)-9,10-secochola-5,7,10(19)-triene-1,3,24a-triol
(5Z,7E)-(1S,3R,24S)-24-(Thien-2-yl)-9,10-secochola-5,7,10(19)-triene-1,3,24a-triol (5Z,7E)-(1S,3R)-1,3-Dihydroxy-24-(Thien-2-yl)-9,10-secochola-5,7,10(19)-trien-24-one
(5Z,7E)-(1S,3R,24S)-24-(4-Methylthiazol-2-yl)-9,10-secochola-5,7,10(19)-triene-1,3,24-triol
(5Z,7E)-(1S,3R,24R)-24-(4-Methylthiazol-2-yl)-9,10-secochola-5,7,10(19)-triene-1,3,24-triol
(5Z,7E)-(1S,3R)-1,3-Dihydroxy-24-(4-methylthiazol-2-yl)-9,10-secochola-5,7,10(19)-trien-24-one
(5Z,7E)-(1S,3R,24S)-24-(Thien-2-yl)-9,10-secochola-5,7,10(19)-triene-1,3,24-triol
(5Z,7E)-(1S,3R,24R)-24-(Thien-2-yl)-9,10-secochola-5,7,10(19)-triene-1,3,24-triol
(5Z,7E)-(1S,3R)-1,3-Dihydroxy-24-(thien-2-yl)-9,10-secochola-5,7,10(19)-trien-24-one
(5Z,7E)-(1S,3R,24S)-24-(4-Methylthien-2-yl)-9,10-secochola-5,7,10(19)-triene-1,3,24-triol
(5Z,7E)-(1S,3R,24R)-24-(4-Methylthien-2-yl)-9,10-secochola-5,7,10(19)-triene-1,3,24-triol
(5Z,7E)-(1S,3R)-1,3-Dihydroxy-24-(4-methylthien-2-yl)-9,10-secochola-5,7,10(19)-trien-24-one
(7E)-(1R,3R,24aR)-24a-Fluoro-24a-(thiazol-2-yl)-24a-homo-19-nor-9,10-secochola-5,7-diene-1,3-diol
(7E)-(1R,3R,24aS)-24a-Fluoro-24a-(thiazol-2-yl)-24a-homo-19-nor-9,10-secochola-5,7-diene-1,3-diol
(5Z,7E)-(1S,3R,24aR)-24a-Fluoro-24a-(thiazol-2-yl)-24a-homo-9,10-secochola-5,7,10(19)-triene-1,3-diol
(5Z,7E)-(1S,3R,24aS)-24a-Fluoro-24a-(thiazol-2-yl)-24a-homo-9,10-secochola-5,7,10(19)-triene-1,3-diol
(7E)-(1R,3R,24aR)-24a-(Acetyloxy)-24a-(thiazol-2-yl)-24a-homo-19-nor-9,10-secochola-5,7-diene-1,3,24a-triol
(7E)-(1R,3R,24aS)-24a-(Acetyloxy)-24a-(thiazol-2-yl)-24a-homo-19-nor-9,10-secochola-5,7-diene-1,3,24a-triol
(7E)-(1R,3R,24aR)-24a-(2,2-Dimethylpropanoyloxy)-24a-(thiazol-2-yl)-24a-homo-19-nor-9,10-secochola-5,7-diene-1,3,24a-triol
(7E)-(1R,3R,24aS)-24a-(2,2-Dimethylpropanoyloxy)-24a-(thiazol-2-yl)-24a-homo-19-nor-9,10-secochola-5,7-diene-1,3,24a-triol
(7E)-(1R,3R)-2-Bromo-24a-(thiazol-2-yl)-24a-homo-19-nor-9,10-secochola-5,7-diene-1,3,24a-triol

Complexes according to the invention that consist of (thiazol-2-yl)-26,27-dinor-9,10-secocholesta-5,7,10(19)-triene-1,3,25-triol with DIMEB, RAMEB or TRIMEB are especially preferred.

A complex that consists of (thiazol-2-yl)-26,27-dinor-9,10-secocholesta-5,7,10(19)-triene-1,3,25-triol with DIMEB is especially preferred.

The complexes according to the invention show a superior stability and durability in comparison to the free and noncomplexed active ingredient.

The production of the complexes according to the invention is carried out with methods that are known in the literature (e.g., A. R. Hedges, Chem. Rev. 1998, 98, 2035-2045; W. Saenger, Angew. Chem. [Applied Chemistry] 42, 343-361 (1980):

1. Co-precipitation method

2. Suspension method

3. Kneading process (paste method)

4. Dry mixing process (dry mixing method)

Soluble complexes can be isolated by freeze-drying.

The production of the cyclodextrin-guest complexes depends on the properties of the guest components, the cyclodextrin and the properties of the complex.

Water-soluble substances can be dissolved—directly or dissolved in an aqueous solvent or solvent mixture—in hot or cold, concentrated aqueous solutions of the cyclodextrin derivative in equimolar amounts or up to a 10-fold excess. Solid complexes that crystallize out immediately or with slow cooling or evaporation, or soluble complexes, which can be isolated by drying processes, can be formed. In this case, the ratio of use of guest components to the cyclodextrin derivative can have an influence on what type of complex is formed. Solid complexes can be filtered off and optionally dried. Soluble complexes can be isolated by drying processes.

Solid complexes and soluble complexes can also be produced simultaneously.

Substances that are not water-soluble are dissolved in, for example, ether, and are layered under or above an aqueous concentrated cyclodextrin derivative solution or shaken therewith.

For industrial production, the kneading process is offered.

Since the complexes of DIMEB according to the invention generally have a lower water-solubility than the DIMEB itself, precipitation methods, for example, are suitable for production. The solubility of the DIMEB is 60 g/100 ml of water (Saenger et al., Langmuir 2002, 18, 5974-5976, Uekama, K.: Irie, T. in Cyclodextrins and their Industrial Uses, Duchene, D., Ed.; Editions Santé, Paris, 1987, p. 395).

The compounds according to the invention with DIMEB are preferably produced by the DIMEB being dissolved in water at a temperature of between 0° and 80° C., and the vitamin D compound or analogs thereof, dissolved in a C1-C10-alcohol such as methanol or ethanol or a C3-C10 ketone, such as acetone or methylethylketone, being added in measured quantities to the aqueous cyclodextrin derivative solution. It is cooled to 0-10° C. for 1-24 hours while being stirred, and the complex is filtered off and optionally dried.

In the complexing, DIMEB can be used in excess with a degree of methylation of 1.8-2.2, whereby generally a 1:1 complex results. Excess DIMEB with a methyl group number that deviates by 14 preferably remains in the solution, by which a purification effect occurs relative to the cyclodextrin derivative. The existing methyl homologs that are caused by production and that deviate from the 2^{nd} degree of methylation are reduced in the complexing. As a result, the molecular weight distribution of the cyclodextrin derivative is more narrow in the complex.

The compounds according to the invention with RAMEB or TRIMEB are preferably produced by concentration by evaporation of the solution, freeze-drying, spray-drying or vacuum drying.

To this end, the vitamin D compound or analogs thereof are added together with the (methylated) cyclodextrin derivative in a soluble form. In this case, both complex components can be dissolved in the same or different solvent(s), preferably aqueous solvents, and then are combined. There is also the possibility, however, first to dissolve one of the components and to add the other in solid form. After 1-24 hours of stirring, the soluble complex, for example, is freeze-dried and obtained as a residue.

The complexes according to the invention are suitable not only for the production of systemically applicable preparations; they can also be used for the production of dosage forms with superior properties that are to be administered topically. The complexes are suitable for preparation of solid and even liquid formulations.

In the storage of preparations, an additional reduction of the active ingredient concentration is often observed as a result of the oxidation as a breakdown pathway of the active ingredient. The durability of the active ingredient complex in the formulation is increased.

It was found for the oral administration that solid preparation forms that contain powdery or crystalline cyclodextrin derivative complexes are advantageous.

The complexes from compound 1 with methylated β-cyclodextrins are not known a priori. These complexes are distinguished compared to complexes that are known a priori in that in the case of topical and systemic administration, a better durability of the active ingredient is ensured. They are distinguished by superior properties; in particular no increased previtamin formation occurs.

The conversion into complexes allows a better manageability and metering capacity of the active ingredient that is active in small dosage units. Also, the complexes according to the invention show a better stability than the noncomplexed substance. For the production of pharmaceutical formulations, the complexes can be mixed with other pharmaceutical adjuvants. The production of the pharmaceutical preparations is in general known a priori.

While native cyclodextrins favor the previtamin formation, methylated β-cyclodextrins in the complexing do not show any additional previtamin formation. In the examples below, a complex, according to the invention, that consists of (thiazol-2-yl)-26,27-dinor-9,10-secocholesta-5,7,10(19)-triene-1,3,25-triol and methylated β-cyclodextrins is described by way of example. Especially with DIMEB, complexes of higher crystallinity are formed, which are advantageously suitable for the production of solid and semisolid formulations. In the described form as a DIMEB complex, the active ingredient has a superior stability compared to the free active ingredient.

In addition to the (thiazol-2-yl)-26,27-dinor-9,10-secocholesta-5,7,10(19)-triene-1,3,25-triol, the natural calcitriol (1α,25-dihydroxyvitamin D₃), as well as a number of structures that are derived from vitamin D, can also be stabilized as a DIMEB complex. The use of DIMEB as a modified cyclodextrin has the advantage that in the production of the complex, no isomerization to the previtamin occurs. These complexes have a superior durability in comparison to the noncomplexed compounds.

In addition, the invention relates to a pharmaceutical composition that contains a complex according to the invention. It can contain additional galenical adjuvants and/or vehicles. The selection thereof depends on the selected dispensing form. In this case, the galenical preparation of the pharmaceutical composition according to the invention can be carried out in a more technical way.

As counterions for ionic compounds, for example, Ca⁺⁺, CaCl⁺, Na⁺, K⁺, Li⁺ or cyclohexylammonium, or Cl⁻, Br⁻, acetate, trifluoroacetate, propionate, lactate, oxalate, malonate, maleinate, citrate, benzoate, salicylate, etc., are suitable. Suitable solid or liquid galenical preparation forms are, for example, granulates, powders, coated tablets, tablets, (micro-)capsules, suppositories, syrups, juices, suspensions, emulsions, drops or solutions for injection (i.v., i.p., i.m., s.c.) or atomization (aerosols), preparation forms for dry-powder inhalation, transdermal systems, as well as preparations with protracted release of active ingredients, in whose production common adjuvants such as vehicles, explosives, binders, coating means, swelling agents, lubricating agents or lubricants, flavoring correctives, sweeteners and solubilizers are used. As adjuvants, for example, magnesium carbonate, titanium dioxide, lactose, mannitol and other sugars; talc, milk protein, gelatin, starch, cellulose and derivatives thereof; animal and plant oils such as liver oil, sunflower oil, peanut oil or sesame oil; polyethylene glycols and solvents, such as, for example, sterile water; and monovalent or multivalent alcohols, for example glycerol, can be mentioned. A pharmaceutical agent according to the invention can thus be produced in that at least one complex that is used according to the invention in a defined dose is mixed with a pharmaceutically suitable and physiologically compatible vehicle and optionally other suitable active ingredients, additives or adjuvants with a defined dose and is prepared in the desired dispensing form.

As diluents, polyglycols, ethanol, water and buffer solutions are suitable. Suitable buffer substances are, for example, N,N′-dibenzylethylenediamine, diethanolamine, ethylenediamine, N-methylglucamine, N-benzylphenethylamine, diethylamine, phosphate, sodium bicarbonate or sodium carbonate. The operation can be performed even without diluents, however.

Physiologically compatible salts, either the vitamin D or vitamin D analog or the cyclodextrin derivative, are salts with inorganic or organic acids, such as hydrochloric acid, sulfuric acid, acetic acid, citric acid, p-toluenesulfonic acid, or with inorganic or organic bases, such as NaOH, KOH, Mg(OH)₂, diethanolamine, ethylenediamine or with amino acids, such as arginine, lysine, glutaminic acid, etc., or with inorganic salts, such as CaCl₂, NaCl or their free ions, such as Ca²⁺, Na⁺, Cl⁻, SO₄²⁻ or combinations thereof. They are produced according to standard methods.

In the pharmaceutical composition, an administration unit can contain 0.1 μg to 1000 μg, preferably 1.0 μg to 500 μg, of the vitamin D compound or the vitamin D analog.

A pharmaceutical composition according to the invention is suitable for prophylaxis and/or treatment of a disease from the group that consists of “diseases that are characterized by hyperproliferation and deficient cell differentiation, in particular hyperproliferative diseases of the skin, such as psoriasis, pituriasis subia pilasis, acne, ichthyosis; pruritus; tumor diseases and precancerous diseases, such as intestinal tumors, breast cancer, lung tumors, prostate cancer, leukemia, T-cell lymphoma, melanoma, beta cell carcinoma, squamous carcinoma, actinic keratoses, cervical dysplasias, metastasizing tumors of any type; diseases that are characterized by disruption of the equilibrium of the immune system, in particular eczemas and diseases of the atopic group; and inflammatory diseases, such as rheumatoid arthritis; respiratory diseases such as asthma; autoimmune diseases such as multiple sclerosis, diabetes mellitus type I, myasthenia gravis, lupus erythematosus, sclerodermia; bullous skin diseases such as pemphigus, pemphigoid; rejection reactions in the case of autologous, allogenic or xenogenic transplants as well as AIDS.” It can be used for treatment and/or prophylaxis of these diseases, whereby one or more administration unit(s) of the pharmaceutical composition is dispensed to an individual who is likely to come down with the disease or who is suffering from it.

### BRIEF DESCRIPTION OF DRAWINGS

FIG. 1 shows the isomerization of Compound 1 in the presence of CD, and

FIGS. 2-4 show x-ray diffraction patterns of crystalline forms of Compound 1.

The examples below represent preferred compositions of the invention without, however, limiting the invention to these examples.

### EXAMPLE 1

Complexing Ergocalciferol and Calcitriol with Native and Methylated β-Cyclodextrins

### 1.1 Complex of Ergocalciferol [(5Z,7E,22E)-(3S)-9,10-Secoergosta-5,7,10(19),22-tetraen-3-ol)] with β-Cyclodextrin (Ratio of Use 1:1)

500 mg (1.26 mmol) of ergocalciferol is dissolved in 3 ml of ethanol and added at 22° C. to 1.43 g (1.26 mmol) of β-cyclodextrin in 80 ml of water. It is stirred for 2 hours at 22° C. and for 2 hours at 0° C. The solid is suctioned off and washed with 5 ml of cold water. After drying, 1.73 g (90% of theory) is obtained as a white solid. After HPLC (Stat. phase Chiracel JHRH 150×4.6 mm ID, detection 244 nm, eluant water/acetonitrile, flow: 1 ml/minute, gradient t₀=70+30 v/v, linear increase of the gradient over 10 minutes t_{10 min} to 60+40 v/v, isocratic t_{10 min-30 min} 60+40 v/v), the solid, in addition to ergocalciferol (t_{Ret.}=26 minutes), contains 24% of the previtamin form (t_{Ret.}=23 minutes).

### 1.2 Complex of Ergocalciferol [(5Z,7E,22E)-(3S)-9,10-Secoergosta-5,7,10(19),22-tetraen-3-ol)] with β-Cyclodextrin (Ratio of Use 1:2)

500 mg (1.26 mmol) of ergocalciferol is dissolved in 3 ml of ethanol and added at 22° C. to 2.86 g (2.52 mmol) of β-cyclodextrin in 40 ml of water. It is stirred for 2 hours while being cooled to 20° C. and stirred for 2 hours at 0° C. The solution is freeze-dried. 3.3 g of product (97% of theory) is obtained. After HPLC (see 1.1), the lyophilizate contains 29% of the previtamin form.

### 1.3 Complex of Ergocalciferol [(5Z,7E,22E)-(3S)-9,10-Secoergosta-5,7,10(19),22-tetraen-3-ol)] with DIMEB (Ratio of Use 1:1)

100 mg (0.25 mmol) of ergocalciferol is dissolved in 0.6 ml of ethanol and added at 22° C. to 335.7 mg (0.25 mmol) of DIMEB in 1 ml of water. It is stirred for 2 hours at 22° C. and for 2 hours at 0° C. After freeze-drying, 445 mg of the ergocalciferol-DIMEB complex is obtained (99.9% of theory). After HPLC (see 1.1), the lyophilizate contains 0.3% of the previtamin form.

### 1.4 Complex of Calcitriol [(5Z,7E)-(1S,3R)-9,10-Secocholesta-5,7,10(19)-triene-1,3,25-triol)] with β-Cyclodextrin (Ratio of Use 1:1.16)

32.7 mg (0.028 mmol) of β-cyclodextrin is dissolved at 30° C. in 2 ml of water under nitrogen atmosphere. 10 mg (0.024 mmol, MW 416.65) of calcitriol (dissolved in 100 μl of ethanol) is added in drops to the clear solution over 10 minutes. It is stirred for 2 hours at 22° C. and for 2 hours at 0-3° C., the solid is filtered off, and it is washed 2 times with 100 μl each of cold water. After drying at 20° C. and 0.5 mbar (24 hours), 26.6 mg of calcitriol-β-CD complex (71.5% of theory) is obtained. After the solid is dissolved in methanol and after an HPLC study (Stat. Phase Chiracel JHRH 150×4.6 mm ID, detection 244 nm, eluant water/acetonitrile, flow: 1 ml/minute, gradient t₀=70+30 v/v, linear increase of the gradient over 10 minutes t_{10 min} to 60+40 v/v, isocratic t_{10 min-30 min} 60+40 v/v), the solid contains 23% of the previtamin form (t_{Ret.}=12 minutes) in addition to calcitriol (t_{Ret.}=17 minutes).

### 1.5 Complex of Calcitriol [(5Z,7E)-(1S,3R)-9,10-Secocholesta-5,7,10(19)-triene-1,3,25-triol)] with DIMEB (Ratio of Use 1:1.16)

38.34 mg (0.028 mmol) of DIMEB is dissolved at 30° C. in 0.25 ml of water under nitrogen atmosphere. 50 μl of ethanol, dissolved [in] 10 mg (0.024 mmol, MW 416.65) of (5Z,7E)-(1S,3R)-9,10-secocholesta-5,7,10(19)-triene-1,3,25-triol, is added in drops to the clear solution. It is stirred for 2 hours at 22° C. and for 2 hours at 0° C., the solid is filtered off, and it is washed 2 times with 50 μl each of cold water. After drying at 20° C. and 0.5 mbar (24 hours), 10.7 mg of solid (42% of theory) is obtained. After the complex is dissolved in methanol and after an HPLC study, the solid contains 0.2% of the previtamin form. After the filtrate is freeze-dried, another 27 mg of lyophilizate is obtained.

Without the presence of β-cyclodextrin, ergocalciferol and calcitriol do not show any significant previtamin formation. With the native β-cyclodextrin, an undesirable isomerization occurs during the complexing. As a complexing agent, however, DIMEB does not induce any isomerization (Table 1).

TABLE 1
Isomerization of Ergocalciferol and Calcitriol

| | Ratio | % Previtamin in the |
|---|---|---|
| | of Use | Isolated Complex |
| Ergocalciferol | | |
| β-Cyclodextrin (1.1) | 1:1 | 24 |
| β-Cyclodextrin (1.2) | 1:2 | 29 |
| DIMEB (1.3) | 1:1 | 0.3 |
| Without Cyclodextrin | — | 0.2 |
| | | (in Solution) |
| Calcitriol | | |
| β-Cyclodextrin (1.4) | 1:1.16 | 23 |
| DIMEB (1.5) | 1:1.16 | 0.2 |
| Without Cyclodextrin | — | 0.3 |
| | | (in Solution) |

### EXAMPLE 2

Complexing of Compound 1 with Native β-Cyclodextrins

### 2.1 Complex with (Thiazol-2-yl)-26,27-dinor-9,10-secocholesta-5,7,10(19)-triene-1,3,25-triol and β-Cyclodextrin (Ratio of Use 1:1.1)

262 mg (0.23 mmol) of β-cyclodextrin is dissolved in 15 ml of water. At 25° C., 100 mg (0.21 mmol) of (thiazol-2-yl)-26,27-dinor-9,10-secocholesta-5,7,10(19)-triene-1,3,25-triol (dissolved in 0.5 ml of ethanol) is added. It is stirred for 2 hours at room temperature, then for 2 hours at 0° C. The solid is filtered off, washed with 1 ml of cold water and dried. 290 mg (85% of theory) of solid is obtained. The content of the solid in the previtamin (compound 2) is 20% (HPLC, Stat. Phase Chiracel JHRH 150×4.6 mm ID, detection 244 nm, eluant water/acetonitrile, flow: 1 ml/minute, gradient to =70+30 v/v, linear increase of the gradient over 10 minutes t_{10 min} to 60+40 v/v, isocratic t_{10 min-30 min} 60+40 v/v, t_{Ref.} Compound 2: 11.65 minutes, t_{Ref.} Compound 1: 18.19 minutes). The sample preparation is carried out by dissolving the complex in 100% methanol.

### 2.2 Complex with (Thiazol-2-yl)-26,27-dinor-9,10-secocholesta-5,7,10(19)-triene-1,3,25-triol and 3-Cyclodextrin (Ratio of Use 1:2.3)

554 mg (0.48 mmol) of β-cyclodextrin is dissolved in 30 ml of water. At 25° C., 100 mg (0.21 mmol) of (thiazol-2-yl)-26,27-dinor-9,10-secocholesta-5,7,10(19)-triene-1,3,25-triol (dissolved in 0.5 ml of ethanol) is added. It is stirred for 2 hours at room temperature, then for 2 hours at 0° C. The solid is filtered off, washed with 1 ml of cold water, and dried. 454 mg of product is obtained as a solid. The HPLC study (see 2.1) of the solid indicates 30% previtamin (compound 2). The sample preparation is carried out by dissolving the complex in 100% methanol.

### 2.3 Complex with (Thiazol-2-yl)-26,27-dinor-9,10-secocholesta-5,7,10(19)-triene-1,3,25-triol and β-Cyclodextrin (Ratio of Use 1:10)

2.38 g (2.1 mmol) of β-cyclodextrin is suspended in 30 ml of water. At 25° C., 100 mg (0.21 mmol) of (thiazol-2-yl)-26,27-dinor-9,10-secocholesta-5,7,10(19)-triene-1,3,25-triol (dissolved in 1 ml of ethanol) is added. It is stirred for 2 hours at room temperature, then for 2 hours at 0° C. The solution is freeze-dried. 2.45 g (98% of theory) of lyophilizate is obtained. The HPLC study (see 2.1) of the lyophilizate indicates 80% of the previtamin form (compound 2). The sample preparation is carried out by dissolving the complex in 100% methanol.

### 2.4 Complex with (Thiazol-2-yl)-26,27-dinor-9,10-secocholesta-5,7,10(19)-triene-1,3,25-triol and γ-Cyclodextrin (Ratio of Use 1:1.1)

100 mg (0.21 mmol) of (thiazol-2-yl)-26,27-dinor-9,10-secocholesta-5,7,10(19)-triene-1,3,25-triol (dissolved in 1 ml of ethanol) is added to 299 mg (0.23 mmol) of γ-cyclodextrin in 1.4 ml of water at 25° C. It is stirred for 2 hours at room temperature, then for 2 hours at 0° C., and the solid is filtered off. After drying, 309 mg (83% of theory) is obtained as a solid. The HPLC study of the solid (see 2.1) indicates 25% of the previtamin form (compound 2).

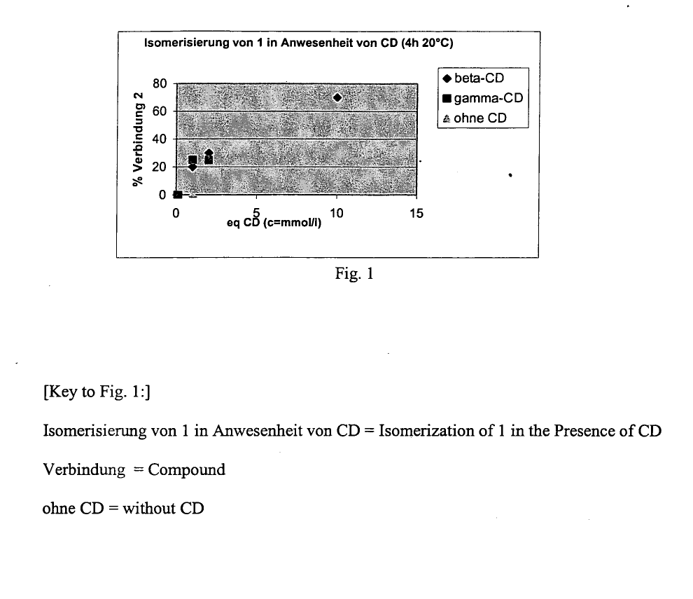

In tests for complexing compound 1 with native β-cyclodextrin (β-CD) and γ-cyclodextrin (γ-CD), an increased previtamin formation to form the compound 2 is observed. The isomerization of compound 1 in the presence of cyclodextrins is compiled in Table 2 and shown graphically in FIG. 1.

### 2.5 Complex with (Thiazol-2-yl)-26,27-dinor-9,10-secocholesta-5,7,10(19)-triene-1,3,25-triol and γ-Cyclodextrin (Ratio of Use 1:2.3)

100 mg (0.21 mmol) of (thiazol-2-yl)-26,27-dinor-9,10-secocholesta-5,7,10(19)-triene-1,3,25-triol (dissolved in 1 ml of ethanol) is added to 633 mg (0.49 mmol) of γ-cyclodextrin in 2.7 ml of water at 25° C. It is stirred for 2 hours at room temperature, then for 2 hours at 0° C., and the solid is filtered off. After drying, 562 mg (85% of theory) of solid is obtained. The HPLC study (see 2.1) of the solid indicates 25% of the previtamin (compound 2).

TABLE 2
Isomerization of Compound 1 in the Presence
of Native β-Cyclodextrins

| | Cyclodextrin | Inner | | |
|---|---|---|---|---|
| | Solubility in | Cavity | Ratio | % |
| | Water (20° C.)* | (A) | of Use | Previtamin |
| Compound 1 | | | | |
| β-Cyclodextrin (2.1) | 1.85 g/100 ml | 6.0-6.4 | 1:1.1 | 20 |
| β-Cyclodextrin (2.2) | | | 1:2.3 | 30 |
| β-Cyclodextrin (2.3) | | | 1:10 | 80 |
| γ-Cyclodextrin (2.4) | 23.3 g/100 ml | 7.5-8.3 | 1:1.1 | 25 |
| γ-Cyclodextrin (2.5) | | | 1:2.3 | 25 |
| Without CD | | — | — | 0.2 (in |
| | | | | Solution) |

*(Source: Szejtly, J., Cyclodextrin Technology, Davies, J. E., Ed; Kluwer Academic Press, Dordrecht, The Netherlands 1988).

### EXAMPLE 3

Complex of Compound 1 with Methylated Cyclodextrins

Cyclodextrin complexes of compound 1 with the cyclodextrins DIMEB, RAMEB and TRIMEB are produced and characterized (Table 3).

### 3 a) Production

### 3 a) 1. Complex that Consists of (Thiazol-2-yl)-26,27-dinor-9,10-secocholesta-5,7,10(19)-triene-1,3,25-triol and DIMEB (Ratio of Use 1:1.2)

1.7 g (1.27 mmol) of heptakis-(2,6-di-O-methyl)-B-cyclodextrin is dissolved in 9 ml of water. At 25° C., 500 mg (1.06 mmol) of (thiazol-2-yl)-26,27-dinor-9,10-secocholesta-5,7,10(19)-triene-1,3,25-triol, dissolved in 1.5 ml of ethanol, is added in drops over a period of 5 minutes. It is stirred for 2 hours at 20-25° C., and it is stirred for another 2 hours while being cooled to 0-5° C. The solid is filtered off and rewashed with a little cold water. It is dried for 24 hours at 20° C. and 15 mbar. 1.9 g (98% of theory) is obtained as a solid.

The XPRD diagram of this solid indicates the presence of a crystalline form (recording of data performed in transmission mode with an automated powder diffractometer with use of germanium-monochromatized CuKα1-radiation (=1.5406 Å); x-ray tubes with a copper anode operated at 40 kV and 30 mA, the 2Θ measurements carried out with the linear site-sensitive detector in the range of 3°≦2Θ≦35° (step width 0.5°). After dissolving in methanol, the solid shows a content (HPLC, p. 2.1) of 25.9% (calc. 26%) of compound 1.

### 3 a) 2. Complex that Consists of (Thiazol-2-yl)-26,27-dinor-9,10-secocholesta-5,7,10(19)-triene-1,3,25-triol and DIMEB (Ratio of Use 1:2.35)

529 mg (0.4 mmol) of heptakis-(2,6-di-O-methyl)-β-cyclodextrin is dissolved in 5 ml of water. At 25° C., 81.4 mg (0.17 mmol) of (thiazol-2-yl)-26,27-dinor-9,10-secocholesta-5,7,10(19)-triene-1,3,25-triol, dissolved in 0.8 ml of ethanol, is added in drops over a period of 5 minutes. It is stirred for 2 hours at 20-25° C. and stirred for another 2 hours while being cooled to 0-5° C. The solid is filtered and rewashed with a little cold water. It is dried for 24 hours at 20° C. and 15 mbar. 300 mg (98% of theory) is obtained. The XPRD diagram (see 3.a) 1.) of this compound shows the presence of a crystalline form.

In the HPLC study (HPLC, p. 2.1), the solid indicates a content of 25.7% (calc. 26%) of compound 1.

### 3 a) 3. Complex with (Thiazol-2-yl)-26,27-dinor-9,10-secocholesta-5,7,10(19)-triene-1,3,25-triol and RAMEB (Ratio of Use 1:1.05)

300 mg (22 mmol) of RAMEB (MW 1303) is dissolved in 2 ml of water. 100 mg (0.21 mmol) of (thiazol-2-yl)-26,27-dinor-9,10-secocholesta-5,7,10(19)-triene 1,3,25-triol is dissolved in 1 ml of ethanol and added to the RAMEB solution. The solution is stirred for 4 hours at 0° C. and then freeze-dried. 390 mg of lyophilizate (100% of theory) is obtained.

### 3 a) 4. Complex with (Thiazol-2-yl)-26,27-dinor-9,10-secocholesta-5,7,10(19)-triene-1,3,25-triol and TRIMEB (Ratio of Use 1:1)

300 mg (0.21 mmol) of TRIMEB (MW 1429.54) is dissolved in 8 ml of water. 100 mg (0.21 mmol) of (thiazol-2-yl)-26,27-dinor-9,10-secocholesta-5,7,10(19)-triene-1,3,25-triol is added, dissolved in 1 ml of ethanol. The solution is stirred for 4 hours and then freeze-dried. 387 mg of lyophilizate (99% of theory) is obtained.

### 3 b) Characterization

### 3 b) 1. X-Ray Diffractometry

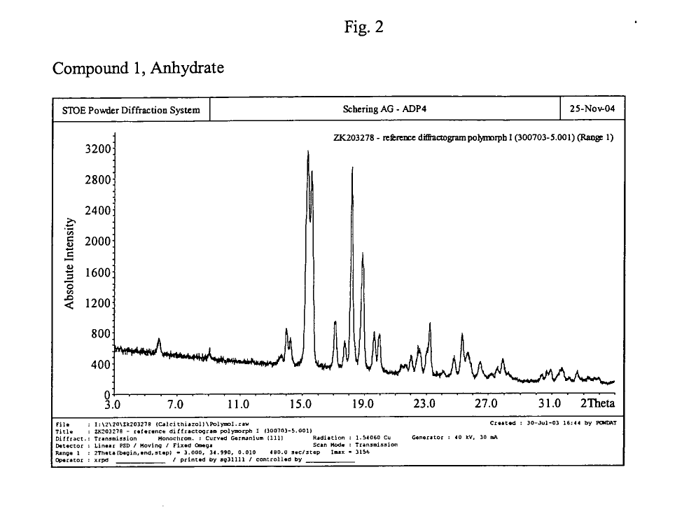

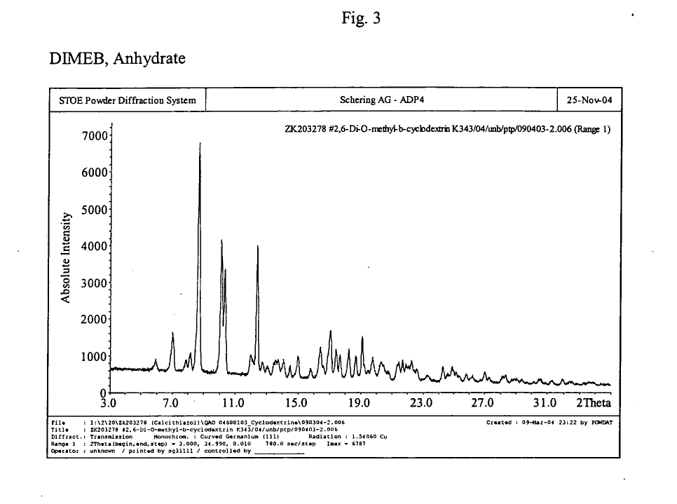

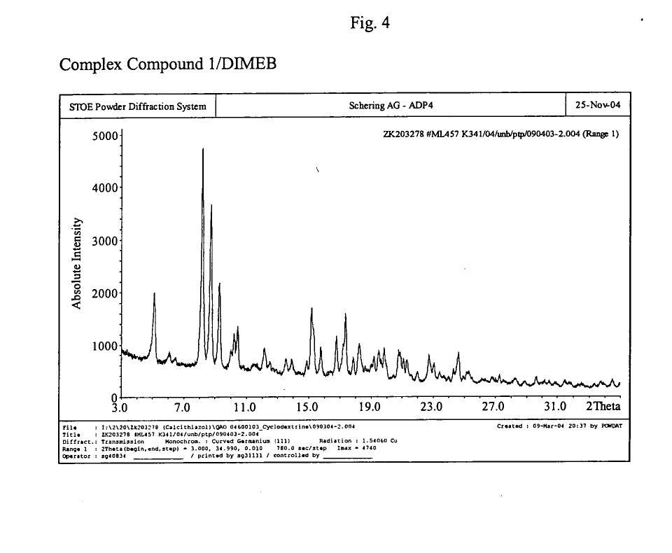

The solid form of the complex is analyzed with x-ray powder diffractometry. In FIGS. 2, 3, and 4, examples of crystalline forms of compound 1, the DIMEB and the complex are shown.

### 3 b) 2. Previtamin Formation

For analytical study, the complexes can be dissolved in methanol or DMF and can be used in these solutions for the HPLC analysis (see 2.1). The content of the complexes that are obtained can be determined in comparison to the pure substance.

The complexes of compound 1 with methylated cyclodextrins are distinguished in that no isomerization in the previtamin takes place.

TABLE 3
Complexes of Compound 1 with Methylated Cyclodextrins
without Additional Previtamin Formation

| | | CD- | | | | |
|---|---|---|---|---|---|---|
| | | Water | | | | |
| | Degree of | Solubility | | % Previtamin in | | |
| | methyl- | (g/100 ml), | Ratio of | the Isolated | | Yield |
| Cyclodextrin | ation | (25° C.) | Use | Complex | Solid Form | (%) |
| Compound 1 | | | | | | |
| DIMEB (3 a) 1) | 2 | 25 | 1:1 | 0.3 | Crystalline | 98 a) |
| DIMEB (3 a) 2) | 2 | 25 | 1:2.35 | 0.2 | Crystalline | 98 a) |
| RAMEB (3 a) 3) | 1.7-1.93 | 10 | 1:1.05 | 0.5 | Amorphous | 100 b) |
| TRIMEB (3 a) 4) | 3 | 200 | 1:1 | 0.3 | Amorphous | 99 b) |
| Without CD | — | — | — | 0.3 | — | — |

a) Yield After Filtration and Drying
b) After Freeze-Drying

### 3b) 3. Stability

In Table 4 below, the stabilities of the DIMEB complex from Example 3 a) 1. and the non-complexed active ingredient are compiled:

Both in solid form (Exp. Nos. 3, 4 and 7 in Table 4) and in aqueous solution (what concentration?), the complex from Example 3a) 1. is more stable than the noncomplexed compound 1 (Exp. Nos. 10-14 in Table 4).

TABLE 4
Stability of the DIMEB Complex from Example 3 a) 1

| | | Complex Purity | | | |
|---|---|---|---|---|---|
| | | from Example | | Compound 1* | Compound 1* |
| Exp. No. | Conditions | 3 a) 1. | By-Products | Purity | By-Products |
| Start¹⁾ | — | 96.44 | 2.61 | 96.12 | 3 |
| 2 | 30 d 40° C. | 96.48% | 2.5% | 96.08 | 3.08 |
| 3 | 3 d/80° C./75% rel F. | 96.25 | 2.86 | 22.55 | 77.45 |
| 4 | 7 d/80° C./75% rel F. | 96.14 | 2.89 | 4.66 | 95.34 |
| 5 | 15 d/40° C./75% rel F. | 96.82 | 2.69 | 96.61 | 2.9 |
| 6 | 15 d/60° C./75% rel F. | 96.69 | 2.81 | 96.34 | 2.96 |
| 7 | 15 d/80° C./75% rel F. | 96.69 | 2.72 | 0 | 100 |
| 8 | 30 d/40° C./75% rel F. | 94.81 | 2.29 | 96.7 | 3.00 |
| 9 | 30 d/60° C./75% rel F. | 94.84 | 2.27 | 96.23 | 2.98 |
| 10 | 15 d/40° C. aqueous *** | 96.55 | 2.81 | 92.78 | 2.92** |
| 11 | 15 d/60° C. aqueous *** | 96.11 | 3.15 | 77.12 | 18.69** |
| 12 | 15 d/80° C. aqueous *** | 96.08 | 3.16 | 3.28 | 96.72** |
| 13 | 30 d/40° C. aqueous *** | 95 | 2.26 | 91.96 | 3.20** |
| 14 | 30 d/60° C. aqueous *** | 94.9 | 2.6 | 59.76 | 37.26** |

*Difference to 100 corresponds to the isomer portion, which is present due to factors related to synthesis as starting contaminants
**Concentration of 20 μg per ml of solution
*** 80 μg of DIMEB complex per ml of solution

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein and of corresponding German application No. 10200501777.5, filed Apr. 13, 2005 and U.S. Provisional Application Ser. No. 60/673,360, filed Apr. 21, 2005 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Complex that consists of a vitamin D compound or a vitamin D analog with a cyclodextrin derivative, whereby optionally the mean molar ratio of vitamin D or vitamin D analog to cyclodextrin derivative is in the range of 1:5 to 5:1, in particular 1:2 to 2:1, and preferably 1:1.2 to 1.2:1.

2. Complex according to claim 1, whereby the cyclodextrin derivative contains n=6 or 7 glucopyranose units.

3. Complex according to claim 1, with a cyclodextrin derivative, in which R1 is a 6-O substituent, R2 is a 4-O substituent, and R3 is a 2-O substituent of the glucopyranose units, whereby R1, R2 and R3 can be the same or different and can be any physiologically compatible radical, but all three cannot be —H at the same time, and are preferably —H, C1-C8-alkyl, linear or branched, saturated or unsaturated, —SO₂OH, —PO(OH)₂, or —CO—R4 with R4=C1-C8-alkyl, whereby the C1-C8-alkyl can be substituted in one or more places, on the same or on different C atoms with —OH, —COOH, —CONHR5, —NHCOR6, —SO₂OH, —PO(OH)₂ or tetrazol-5-yl, with R5=—H or C1-C4-alkyl and R6=carboxyphenyl, whereby R1, R2 and R3 can be randomized in different glucopyranose units, whereby an oxygen atom or several oxygen atoms of the glucopyranose units, in particular the oxygen atom 6-O, can be replaced by sulfur atoms, or with a physiologically compatible salt of such a cyclodextrin derivative.

4. Complex according to claim 1, with a cyclodextrin derivative, whereby n=7 and R1, R2, and R3 are the same or different, but all three are not —H at the same time, and are —H or C1-C8 alkyl or C1-C8 hydroxyalkyl.

5. Complex that consists of vitamin D compounds or analogs thereof with a 5Z,7E,10(19)-triene system and methylated derivatives of the β-cyclodextrin.

6. Complex according to claim 1, whereby the complex, as a vitamin D analog, contains a compound according to general formula I 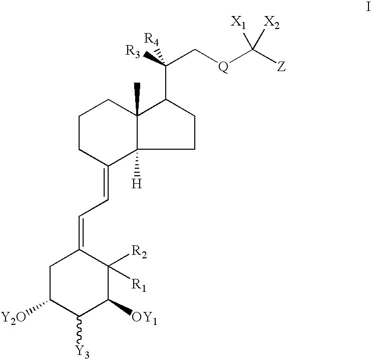
in which
Y₁ and Y₂, independently of one another, each mean a hydrogen atom or a group —C(O)R₅,
and Y₃ means a hydrogen atom or a hydroxy group, a halogen atom, a group —OC(O)R₅ or an OR₅ group,
whereby
R₅ stands for an aromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms, and/or 1-2 NH groups and/or optionally is substituted by 1-2 hydroxy groups, 1-2 amino groups, 1-2 SH groups, 1-2 COOH groups and/or 1-2 phenyl groups,
and the group Y₃ can be present both in the 2α-situation and in the epimeric 2β-situation,
R₁ and R₂ together mean an exocyclic methylene group,
R₃ and R₄, independently of one another, each mean a hydrogen atom, a fluorine, chlorine or bromine atom, an alkyl group with 1 to 4 carbon atoms, together a methylene group, or, together with the quaternary carbon atom 20, a 3- to 7-membered, saturated or unsaturated carbocyclic ring,
Q means a straight-chain alkylene group with 1 to 5 carbon atoms,
X₁ and X₂ together mean a double-bonded keto-oxygen atom or, independently of one another, a hydrogen atom, a hydroxy group, an —O(CO)R₅ group, a fluorine, chlorine or bromine atom,
whereby X₁ and X₂ should each be, not simultaneously, a hydroxy group or an —O(CO)R₅ group,
Z means a carbocyclic or heterocyclic, optionally aromatic or heteroaromatic ring with 5 or 6 ring members or a condensed ring system that consists of a 5- and a 6-membered ring or two 6-membered rings, which can be substituted by one or more fluorine, chlorine, bromine or iodine atoms, one or more hydroxy groups, one or more COOR₆ groups, one or more C₁-C₅-alkyl groups, which in turn can be substituted by one or more fluorine, chlorine, bromine, or iodine atoms, C₁-C₆-alkoxy groups and/or COOR₆ groups,
whereby
R₆ stands for a C₁-C₆-alkyl group, a benzyl group or a phenyl group,
as well as all possible epimers or diastereomers and mixtures thereof.

7. Complex according to claim 1, whereby the complex, as a vitamin D analog, contains (thiazol-2-yl)-26,27-dinor-9,10-secocholesta-5,7,10(19)-triene-1,3,25-triol.

8. Complex according to claim 1, whereby the complex, as a vitamin D compound or analog thereof, contains calcitriol, ergocalciferol, calcipotriol, tacalcitol, 25-hydroxy-vitamin D₃, or 1α-hydroxycholecalciferol.

9. Complex according to claim 1, whereby the complex, as a methylated β-cyclodextrin derivative, contains DIMEB.

10. Complex according to claim 9, characterized in that the DIMEB has a degree of methylation of more than 1.8 and of less than 2.2.

11. Complex according to claim 1, whereby the complex, as a methylated β-cyclodextrin derivative, contains RAMEB or TRIMEB.

12. Complex that consists of (thiazol-2-yl)-26,27-dinor-9,10-secocholesta-5,7,10(19)-triene-1,3,25-triol and DIMEB, RAMEB or TRIMEB.

13. Process for the production of a complex according to claim 1, wherein
a) the preferably methylated derivative of the cyclodextrin, preferably the β-cyclodextrin, is dissolved at a temperature of between 0° and 80° C. in water, and the vitamin D compound or analog thereof, dissolved in methanol, ethanol, a C1-C10-alcohol or a C3-C10 ketone, such as, e.g., acetone, or methylethylketone, is added in measured quantities to the aqueous cyclodextrin derivative solution,
b) is stirred for 1-24 hours while being cooled at 0-30° C.,
c) the solid that is obtained is filtered off, and
d) optionally is dried.

14. Process according to claim 13, wherein the vitamin D compound, or analogs thereof, is dissolved in ethanol.

15. Process according to claim 13, wherein as a methylated derivative of the β-cyclodextrin, DIMEB is used.

16. Process for the production of a complex according to claim 1, wherein
a) the methylated derivative of the β-cyclodextrin is added together with the vitamin D compound or analogs thereof in a soluble form,
b) is stirred for 1-24 hours, and
c) the complex is obtained from the solution by concentration by evaporation of the solution, freeze-drying, spray-drying or vacuum-drying.

17. Process for the production of a complex according to claim 16, wherein as a methylated derivative of β-cyclodextrin, RAMEB or TRIMEB is used.

18. Process according to claim 15, wherein the two complex components are dissolved in the same or different solvent(s), preferably aqueous solvents, and then are combined, or wherein one of the components is dissolved, and the other is added in solid form to this solution.

19. Process for the production of a complex according to claim 1, wherein
a) the methylated derivative of β-cyclodextrin and the vitamin D compound are added together in soluble form,
b) are stirred for 1-24 hours,
c) the optionally obtained solid is filtered off and optionally dried,
d) the complex is obtained from the filtrate (=after the solution that is separated from the solid is filtered) by concentration by evaporation of the filtrate, freeze-drying, spray-drying or vacuum-drying.

20. Pharmaceutical composition that contains a complex according to claim 1.

21. Pharmaceutical composition according to claim 20, in addition containing galenical adjuvants and/or vehicles.

22. Pharmaceutical composition according to claim 20, whereby an administration unit contains 0.1 μg to 1000 μg, preferably 1.0 μg to 500 μg, of the vitamin D compound or the vitamin D analog.

23. Use of a complex according to claim 1 for the production of a pharmaceutical composition, whereby optionally the complex is mixed with one or more galenical adjuvants and/or vehicle(s) and is administered in a dispensing form to the administration units.

24. Use according to claim 23, whereby the pharmaceutical composition for prophylaxis and/or treatment of a disease is prepared from the group that consists of “diseases that are characterized by hyperproliferation and deficient cell differentiation, in particular hyperproliferative diseases of the skin, such as psoriasis, pituriasis subia pilasis, acne, ichthyosis; pruritus; tumor diseases and precancerous diseases, such as intestinal tumors, breast cancer, lung tumors, prostate cancer, leukemia, T-cell lymphoma, melanoma, beta cell carcinoma, squamous carcinoma, actinic keratoses, cervical dysplasias, metastasizing tumors of any type; diseases that are characterized by disruption of the equilibrium of the immune system, in particular eczemas and diseases of the atopic group; and inflammatory diseases, such as rheumatoid arthritis, respiratory diseases such as asthma; autoimmune diseases such as multiple sclerosis, diabetes mellitus type I, myasthenia gravis, lupus erythematosus, sclerodermia; bullous skin diseases such as pemphigus, pemphigoid, rejection reactions in the case of autologous, allogenic or xenogenic transplants as well as AIDS.”

25. Use of a complex according to claim 1, or a pharmaceutical composition, for prophylaxis or treatment of a disease, whereby one or more administration unit(s) of the pharmaceutical composition is dispensed to an individual who is likely to come down with the disease or who is suffering from it.
